(19) United States
(12) Patent Application Publication
(10) Pub. No.: US 2004/0131578 A1
(43) Pub. Date: Jul. 8, 2004

(54) Antibacterial shaving foam/gel formulations

(75) Inventor: Navin Geria, Warren, NJ (US)

Correspondence Address: Richard R. Michaud, McCormick, Paulding & Huber LLP, CityPlace II, 185 Asylum Street, Hartford, CT, 06103, US

(73) Assignee: Eveready Battery Company, Inc., Westlake, OH

(21) Appl. No.: 10/686,995

(22) Filed: Oct. 16, 2003

Related U.S. Application Data
(60) Provisional application No. 60/420,274, filed on Oct. 21, 2002.

(51) Int. Cl. A61K 7/15

(52) U.S. Cl. 424/73

(57) ABSTRACT

The present invention provides shaving gel and foam formulations comprising dichlorobenzyl alcohol as an antibacterial agent.

### CROSS-REFERENCE TO RELATED APPLICATIONS

This application is entitled to the benefit of and incorporates by reference essential subject matter disclosed in Provisional Patent Application No. 60/420,274 filed on Oct. 21, 2002.

### FIELD OF THE INVENTION

The present invention relates to antibacterial shaving foam and gel formulations comprising dichlorobenzyl alcohol as an antibacterial agent

### BACKGROUND OF THE INVENTION

The compound Triclosan®, i.e., 5-chloro-2-(2,4-dichlorophenoxy)phenol (formerly marketed as Irgasan®; Ciba Geigy Chemical Corp.), is one of the most popular antibacterial ingredients for topically administered, human personal health care products. Recently, however, European Union member associations have ruled that, until environmental safety information supporting the continued use of Triclosan® has been gathered and evaluated completely, there should be no new introduction of Triclosan®-based “down-the-drain” personal health care products. This ruling is based primarily on the observation that Triclosan® has a chemical structure similar to known environmental toxins, including dioxins and PCB's. Accordingly, a continued need exist for alternative antibacterial products in the personal health care field, especially those involving shaving foams and gels.

The instant invention provides shaving gel and foam formulations comprising dichlorobenzyl alcohol as an antibacterial agent. The use of dichlorobenzyl alcohol as an antibacterial agent in limited classes of personal health care products is generally known. For example, U.S. Pat. Nos. 4,167,583 and 3,123,528; PCT International Application Publications No. WO 97/46218; and British Pat. No. GB 906,870 disclose the use of certain regioisomers of dichlorobenzyl alcohol as microbiocidally active ingredients in lotion, ointment, and cream formulations for topical administration. Additionally, the use of dichlorobenzyl alcohol as a preservative in topical product formulations is also generally known. See, for example, U.S. Pat. Nos. 5,851,543, 5,804,203, 5,738,856, and 5,470,017.

The present invention provides antibacterial shaving gel and foam formulations comprising dichlorobenzyl alcohol as an antibacterial agent. The use of dichlorobenzyl alcohol as an antibacterial agent is especially advantageous in the practice of the present invention as it affords potent topical antibacterial benefits over an extended period of time, is well-tolerated and non-sensitive to the skin, and does not present the potential for long-term adverse environmental effects. Furthermore, dichlorobenzyl alcohol exhibits good stability in high pH shaving foam and gel formulations, and is chemically compatible with anionic, cationic, non-ionic, and amphoteric surfactants, as well as additional preservatives, biocides, and other adjuvants commonly employed in such formulations.

### SUMMARY OF THE INVENTION

The present invention provides shaving gel and foam formulations comprising dichlorobenzyl alcohol as an antibacterial agent.

### DETAILED DESCRIPTION OF THE INVENTION

The present invention provides antibacterial shaving gel and foam formulations comprising dichlorobenzyl alcohol as an antibacterial agent

The term “dichlorobenzyl alcohol”, as employed throughout the instant description and appendant claims, is intended to embrace any, and/or all, regioisomeric configurations of dichlorobenzyl alcohol, i.e., 2,3-dichloro-, 2,4-dichloro-, 2,6-dichloro-, 3,4-dichloro-, and 3,5-dichlorobenzyl alcohol.

The present shaving formulations may comprise the form of creams, or may be dispensed from a suitable package as an aerosol foam or a post-foaming gel. The present invention is particularly concerned with aerosol foams and post-foaming gels that can be dispensed from a valved container adapted to maintain the product under pressure and dispense it when desired by opening the valve. Preparation of both foam and gel formulations for dispensation may be prepared according to known methods, for example, according to those methods disclosed in U.S. Pat. No. 5,560,859, and 5,902,778, the disclosures of which are incorporated herein by reference. Aerosol-foam shaving compositions are normally contained in a single-compartment dispenser with the inert volatile liquid agent as the propellant gas in accordance with conventional aerosol technology.

Post-foaming gel shaving compositions can be packaged in many types of commercially available containers. Where aerosol dispensers are employed, generally the gel is maintained in a package separate from the propellant by means of a ‘bag-in-can’ packaging system, or a diaphragm inside the package driven by a propellant, or a mechanical force, such as a spring. Suitable dispenser forms are described in, e.g., U.S. Pat. No. 3,541,581, the disclosure of which is incorporated herein by reference.

Thus, according to the invention, there are provided antibacterial compositions intended for shaving of the skin comprising from about 40 to about 90% by weight of water, from about 4% to about 25% by weight of water-soluble soap, from about 0.5% to about 12% by weight of an inert volatile liquid agent and from about 0.1% to about 5.00% by weight of dichlorobenzylalcohol.

The essential components of the composition according to the invention may be combined with tap water, distilled water or deionised water. The composition should comprise from about 40% to about 90% by weight of water, more preferably from about 50% to about 80% by weight of water. The upper limit of water in the composition maybe determined by the maximum amount that will produce a satisfactory gel or foam in use. In addition, the composition may optionally small amount of polar substances, such as low molecular weight alcohols, methanol, ethanol, propanol and isopropanol, provided that these do not detract from the overall properties of the composition. These may generate a cooling effect on the skin during the shaving process.

The soap used in the formulations of the invention may comprise any conventional soap known to one of ordinary skill in the relevant art. Generally preferred soaps include water-soluble stearate and palmitate soaps, such as potassium, ammonium, sodium, and the soluble amine soaps of commercial stearic and palmitic acid. Such soaps may typically be prepared by neutralization of the appropriate higher fatty acid with suitable alkali, or may be introduced in the form of animal or vegetable fats which are rich in the appropriate acid, and which, when saponified, form soaps rich in the corresponding acid. Mixtures of various soaps may also be used. In general, lathers comprising animal fat and vegetable fat soaps are preferred. Preferably from about 30% to about 100% by weight of the soap used in the composition comprises a water-soluble stearate, more preferably from about 60% to about 100% by weight of the soap comprises a water-soluble stearate.

The composition according to the invention may optionally comprise a wetting agent. Typical wetting agents include triethanolamine lauryl sulfate, sodium lauryl sulfate, sodium dodecyl benzene sulfonate water-soluble polyoxyethylene cetyl ethers of alkyl substituted phenols, and water soluble polyoxyethylene ethers of alkyl substituted phenols and water-soluble polyoxyethylene cetyl ethers. The wetting agent in the composition may be effective in facilitating the rinsing of the lather from the skin. If used, preferably the wetting agent is present in the composition at a level of from about 0.5% to about 6% by weight. Preferably, the total amount of soap (including any wetting agent used in the composition according to the invention is from about 4% to about 25% by weight, more preferably, from about 5% to about 20% by weight, and most preferably from about 10% to about 18% by weight of the total composition.

The composition may optionally comprise from about 0.01% to about 5% by weight of a water-soluble gelling agent. Gelling agents optionally employed in compositions of the post-foaming gel type of the present invention are typically water-soluble derivatives of naturally occurring substances such as cellulose, sucrose and glucose. Preferred gelling agents include the co-polymers of acrylic acid and polyallyl sucrose; the reaction products of cellulose with acids; the reaction products of glucose with acids; the reaction products of cellulose with alkaline oxides; and the reaction products of glucose with alkaline oxides. The optional gelling agents may be used in the composition at a level of about 0.01% to about 5% by weight, more preferably from about 0.01% to about 2.5% by weight, most preferably from about 0.05% to about 1.5% by weight of the total composition. Suitable acrylate derivatives include the Carbopol range of polymers. Cellulose derivatives are, however, preferred, since they afford good lubrication for the shaving blade, as well as functioning as a gelling aid. Suitable cellulose derivatives include sodium carboxymethyl cellulose, cellulose methyl ether, and hydroxy alcohol cellulose. Particularly preferred cellulose derivatives are the commercially available Natrosol and Klucel. These may typically be used at a level of from about 0.01% to about 0.4% by weight of the composition.

The inert volatile liquid agent used in the aerosol form of the composition of the present invention should be suitable to function as an aerosol propellant gas, and can be selected from a wide variety of the propellants known in the industry. Suitable selections include inert inorganic gases such as carbon dioxide, nitrogen, argon, and air. However, in general, more uniform foaming is achieved by a selection from lower hydrocarbon, or wholly or partially halogenated hydrocarbon liquefied propellants. Such are usually emulsified in the aqueous phase of the compositions. Suitable examples of liquid or liquefied propellent agents include saturated aliphatic hydrocarbons having from 1 to 4 carbon atoms. They can be unhalogenated, such as propane, n-butane and iso-butane, or halogenated with fluorine or chlorine atoms, such as monocholorotrifluoromethane, dichlorodifluromethane, trichloromonofluromethane, and similar chloroflurohydrocarbons preferably having from 1 to 3 carbon atoms. Alternatively, mixtures of hydrocarbons and halogen-substituted hydrocarbons maybe used.

The post-foaming gels of the present invention may further comprise a water-soluble gelling agent, and an inert volatile liquid agent as a post-foaming agent. Such gels should be appropriately packaged such that when the gel is stored for prolonged periods, it remains as a stable homogenous composition. However, on being dispensed, it should remain substantially free of foaming until it is appropriately activated, for example by manual shearing. Preferably the gel composition has a yield value sufficiently high to substantially restrain the composition from foaming for at least 60 seconds under static ambient conditions.

The post-foaming gels of the present invention preferably comprise coherent colloidal dispersions comprising at least four base components, including water, a gelling agent, soap, and a post-foaming agent, in addition to the dichlorobenzyl alcohol. They will generally exhibit mechanical properties characteristic of the solid state, and will comprise a matrix of homogeneously dispersed components and medium. Typically, the gels of the invention have a yield value, i.e., they resist flow up to a given sheering tension, and then behave as an elastic solid below that tension. They are typically formed from a solution, as opposed to being formed from a solid substance which exhibits swelling power. In order that stable gel can be formed from a solution, it is necessary that a solid substance separates from a solution in a finely dispersed colloidal state, and the separated solid particles are neither deposited by gravity, nor remain in a colloidal suspension as freely moving kinetic units. Rather, they join together to form a continuous coherent framework, throughout the mass of the solution.

As the post-foaming agent for use in the gel compositions of the present invention, the inert volatile liquid should be capable of being dispersed continuously throughout the stable gel, and compatible with other components of the gel. The vapor pressure of the post foaming agent is also important, in that lather should be produced by volatilization of the post-foaming agent when the gel is rubbed between the fingers, or on the skin. Additionally, when the gel is dispensed from the storage container, it should remain free from foaming for at least 60 seconds.

Suitable post-foaming agents for use in the gel compositions of the invention comprises liquids or are liquefiable, and include saturated aliphatic hydrocarbons having from 4-6 carbon atoms, such as butane, pentane and hexane (in particular is opentane and isobutene). Other suitable post-foaming agents include partially, or wholly halogenated hydrocarbons, such as trichlorofluroethane. Also, mixtures of aliphatic and halogenated hydrocarbon propellants, or post-foaming agents can be used. Generally suitable post-foaming agents are those substances that have a low solubility in water, for example less than about 20 cc of gas in 100 grams of water at one atmosphere and 20° C.

The amount of inert volatile agent used in the compositions of the present invention may have an important effect on the properties of the composition, including the stability of foam, the yield value, post-foaming characteristics of the gel compositions, and the foam profile. The amount of volatile agent, whether as propellent or post-foaming agent may, however, routinely be varied by one of ordinary skill in the art to optimize the desired characteristics of the gel or the foam.

Aliphatic hydrocarbon volatile agents typically comprise from about 0.5% to about 4% by weight of the composition, more preferably from about 1.5% to about 3.5% by weight. Halogenated hydrocarbon agents would typically comprise from about 1% to about 12% by weight, more preferably, from about 1% to about 8% by weight and, most preferably, from about 3% to about 7% by weight of the composition.

### EXAMPLES

The shaving gel composition according to the following composition was prepared, and tested for its antimicrobial properties.

### Example 1

| Ingredients | % w/w |
|---|---|
| Propylene Glycol | 1.800 |
| Palmitic acid | 7.900 |
| Stearic acid | 2.900 |
| Ceteth-2 (Brij52) | 0.790 |
| Triethanolamine 98% | 6.200 |
| Dichlorobenzyl alcohol | 2.00 |
| Fragrance | 0.50 |
| Glycerine | 2.500 |
| Sodium Isostearoyl Lactylate | 0.200 |
| Hydroxyethyl cellulose | 0.25 |
| Hydroxypropyl cellulose | 0.300 |
| Isopentane/Isobutane | 3.00 |
| Aloe Barbedensis | 0.500 |
| Deionized water | to 100.00 |

The composition maybe manufactured by weighing the mixing vessel and adding glycerine. The hydroxyethyl and propylcellulose are added with stirring, followed by aloe and dichlorobenzyl alcohol, with mixing until homogeneous. One-third of the deionised water is also slowly added.

To the main mixing tank, one-third water is added and heated to 80° C. To this is added stearic and palmitic acid with stirring, until the mass has melted. The batch is maintained at 80° C. and the Ceteth-2 and propylene glycol are then added.

The remaining ingredients, except for the fragrance and the is opentane/isobutene, are pre-mixed in another container, and then added to the main mixing vessel with agitation. The temperature is maintained at 80° C. until the mixture is homogeneous. The contents are then cooled to 35° C. with stirring, the perfume, if desired, is added and the contents mixed until the batch is homogeneous. The batch is then filled with the is opropane/isobutene gas into appropriate cans, in a conventional manner.

### Example 2

The following compositions represent post foaming shaving gels according to the invention, which can be prepared according to a method analogous to that described in Example 1.

| Ingredients | % w/w | % w/w |
|---|---|---|
| Propylene glycol | 1.500 | 1.500 |
| Palmitic acid | 8.00 | 8.00 |
| Stearic acid | 2.500 | 2.500 |
| Propylene glycol Isostearate | 1.00 | 0.700 |
| Ceteth-2 | 1.0 | 1.00 |
| Triethanolamine 98% | 5.800 | 5.800 |
| Dichlorobenzyl Alcohol | 0.100 | 5.00 |
| Fragrance | 0.50 | 0.50 |
| Propylene glycol | 1.0 | 1.0 |
| Hydroxy ethyl cellulose | 0.25 | 0.25 |
| Hydroxy propyl cellulose | 0.02 | 0.02 |
| Isopropane/Isobutane | 3.0 | 3.0 |
| FD&C Blue No. 1 | trace | trace |
| Deionised Water | to 100.00 | 100.00 |

### Example 3

The following examples represent two aerosol shaving foams according to the invention, which can be prepared according to a method analogous to that described in Example 1.

| Ingredients | % w/w | % w/w |
|---|---|---|
| Stearic acid | 3.500 | 3.500 |
| Palmitic acid | 3.500 | 3.500 |
| Lauric acid | 0.50 | 0.50 |
| Dichlorobenzyl alcohol | 3.0 | 4.00 |
| Fragrance | 0.50 | 0.50 |
| Potassium Hydroxide (50% aq. Solution) | 0.60 | — |
| Cocamidopropyl Betaine | 0.60 | 0.60 |
| Glycerine | 5.10 | 5.10 |
| Mineral oil | 1.90 | 1.30 |
| Procetyl AWS | 0.10 | 0.10 |
| Menthol | 0.20 | 0.20 |
| Butylated Hydroxy Toluene | 0.05 | 0.05 |
| Sodium Isostearoyl Lactylate | 0.20 | 0.20 |
| Butane/isobutene/propane mix | 5.0 | 5.0 |
| Deionised water | to 100.0 | 100.00 |

Antibacterial Testing

Determination of the antibacterial properties of the shaving foams and gels of the instant invention over predetermined periods of time was performed according to ASTMStandard E 1891-97, Standard Guide for the Determination of a Survival Curve for Antimicrobial Agents Against Selected Microorganisms and Calculation of a D-Value and Concentration Coefficient.

The test organisms used in the determinations were Staphylococcus aureus, a common skin bacteria that can be pathogenic under certain circumstances, and Pseudomonas aeruginosa, a bacteria responsible for a diversity of infections. The time limit set for the test was approximately two minutes, which represents an average time that the shaving foam or gel would be present on the surface of the skin during shaving. The bacteria were inoculated into about 1 g of the product, which was then mixed to a smooth foam consistency. After about two minutes, the product was plated using standard dilution and plating techniques. Following incubation of the plates, surviving bacteria were enumerated. Reductions of between 80.6% and 86.3% of the Staphylococcus aureus, and at least 99.95% of the Pseudomonas aeruginosa were observed.

What is claimed is:

1. A shaving composition comprising from about 40% to about 90% by weight of water; from about 4% to about 25% by weight of a water-soluble soap; from about 0.5% to about 12% by weight of an inert volatile liquid agent; and from 0.1% to about 5.00% of dichlorobenzyl alcohol.

2. A shaving composition according to claim 1 which is an aerosol shaving foam composition, wherein said volatile liquid agent comprises an aerosol propellant gas.

3. A shaving composition according to claim 1 further comprising from about 0.01% to about 5.00% by weight of water-soluble gelling agent.

4. A shaving composition according to claim 3 which is a post-foaming shaving gel comprising of from about 0.01% to about 5% by weight of said water-soluble gelling agent and wherein the volatile liquid agent is a post-foaming agent.

5. A shaving composition according to claim 4 wherein the gelling agent is selected from the group consisting of co-polymers of acrylic acid and polyallyl sucrose; the reaction products of cellulose with acids; the reaction products of glucose with acids; the reaction products of cellulose with alkaline oxides; and the reaction products of glucose with alkaline oxides.
